(19) United States
(12) Patent Application Publication
(10) Pub. No.: US 2018/0256033 A1
(43) Pub. Date: Sep. 13, 2018

(54) TOOTH CRACK OBSERVATION TIP COUPLABLE TO LIGHT CURING DEVICE

(71) Applicant: DXM CO., LTD, Goyang-si, Gyeonggi-do (KR)

(72) Inventors: Du Rok JUNG, Goyang-si, Gyeonggi-do (KR); Kyoung Soo SHIN, Goyang-si, Gyeonggi-do (KR)

(73) Assignee: DXM CO., LTD, Goyang-si, Gyeonggi-do (KR)

(21) Appl. No.: 15/760,921

(22) PCT Filed: Sep. 2, 2016
(86) PCT No.: PCT/KR2016/009837

§ 371 Date: Mar. 16, 2018

(30) Foreign Application Priority Data
Sep. 21, 2015 (KR) 10-2015-0132765

(51) Int. Cl. A61B 5/00; A61C 13/15; G02B 5/22

(52) U.S. Cl. CPC A61B 5/0088; G02B 5/226; A61C 19/004

(57) ABSTRACT

Provided is a tooth crack observation tip comprising: a tip body inserted in a coupling tube of a light curing device from which a light guide tip has been separated; a crack observation guide tube formed to extend from the tip body by a predetermined length and inserted into the mouth; and a light conversion filter which is coupled to a rear end of the tip body and converts blue light into white light, the light conversion filter being formed by coating a red color and a green color on both surfaces of glass thereof, respectively.

### TECHNICAL FIELD

The present invention relates to a tooth crack observation tip, and more particularly, to a tooth crack observation tip that is separably coupled to a light curing device so as to detect dental caries or cracks.

### BACKGROUND ART

Light-cured polymer is used for medical materials such as resins, which are used in dental treatment or dental technology. In order to harden light-cured polymer, a light curing device is used. An exemplary conventional light curing device is disclosed in Korean Patent Registration No. 10-0547259, entitled “LED HAVING WIDE WAVELENGTH RANGE AND LIGHT CURING DEVICE USING THE SAME”.

FIG. 1 is a view illustrating an example of a conventional light curing device 10. As illustrated, the conventional light curing device 10 is provided therein with a light source, which is configured to generate blue light L1. A light guide tip 20 for guiding the blue light L1 generated inside the light curing device 10 to an oral cavity is coupled to the front end of the light curing device 10. The light guide tip 20 includes a light guide pipe 21 coupled to the light curing device 10 and a diffusion pipe 23 provided at the front end of the light guide pipe 21 in order to diffuse light into an oral cavity.

Here, the light guide pipe 21 has a diameter d1 capable of emitting light in a wide range r1 within the oral cavity.

Recently, with growing public interest in dental caries, the number of patients who visit dental clinics to receive treatment for dental caries is increasing. In order to detect dental caries, the “METHOD AND APPARATUS FOR DETECTING DENTAL CARIES”, which is disclosed in Korean Patent Laid-open Publication No. 10-2011-0105836, are being used.

However, the conventional dental crack observation apparatus disclosed in the above document has a complicated structure and must be separately purchased, which increases dental equipment installation costs.

### DISCLOSURE

### Technical Problem

Therefore, the present invention has been made in view of the above problems, and it is an object of the present invention to provide a tooth crack observation tip that is separably coupled to a light curing device, which is widely used in dental clinics, thereby detecting dental caries or cracks conveniently.

It is another object of the present invention to provide a tooth crack observation tip that has a simple structure and can be easily manipulated, thereby providing improved convenience in use.

The above objects and other advantages of the present invention will be more clearly understood by those skilled in the art from a preferred embodiment of the present invention set forth herein.

### Technical Solution

In accordance with an aspect of the present invention, the above and other objects can be accomplished by the provision of a tooth crack observation tip configured to be separably coupled to a light curing device for generating blue light and guiding the blue light to a light guide tip. The tooth crack observation tip includes a tip body configured to be inserted into a coupling pipe of the light curing device from which the light guide tip has been separated, a crack observation guide pipe extending a predetermined length from the tip body, the crack observation guide pipe being configured to be inserted into an oral cavity, and a light conversion filter coupled to the rear end of the tip body, the light conversion filter being configured to convert the blue light into white light. The light conversion filter is formed by glass having two opposite surfaces that are colored red and green, respectively.

According to an embodiment, the tip body may be formed to have an outer diameter that corresponds to the inner diameter of the coupling pipe, and may have a finger insertion recess depressed to a predetermined depth from the outer circumferential surface thereof so as to allow fingers to be inserted thereinto.

According to an embodiment, the crack observation guide pipe may be formed of glass fiber, and may have a diameter that ranges from a twentieth to a tenth of the diameter of a light guide pipe of the light guide tip.

In accordance with another aspect of the present invention, there is provided a tooth crack observation tip configured to be separably coupled to a light curing device for generating blue light and guiding the blue light to a light guide tip. The tooth crack observation tip includes a tip body configured to be inserted into a coupling pipe of the light curing device from which the light guide tip has been separated, and a crack observation guide pipe extending a predetermined length from the tip body, the crack observation guide pipe being configured to be inserted into an oral cavity. The crack observation guide pipe has a rear end that is coupled to the tip body, the rear end having two opposite surfaces that are colored red and green, respectively, so as to convert the blue light into white light.

### Advantageous Effects

The tooth crack observation tip according to the present invention can be used in the state of being separably coupled to a light curing device. As a result, it is possible to realize all of a photopolymerization function, a crack observation function and a dental caries detection function using a single light curing device.

In addition, the tooth crack observation tip of the present invention has a simple structure so as to be easily inserted into or separated from a light curing device by a dental operator, and emits white light in a relatively narrow range, whereby a dental operator is capable of detecting the presence of dental cracks or caries with the naked eye.

### DESCRIPTION OF DRAWINGS

FIG. 1 is a view illustrating the structure of a conventional light curing device;

FIG. 2 is a side view illustrating the state in which a tooth crack observation tip according to the present invention is coupled to a light curing device;

FIG. 3 is an exploded perspective view illustrating the process of coupling the tooth crack observation tip according to the present invention to the light curing device;

FIG. 4 is a cross-sectional view illustrating the state in which the tooth crack observation tip is coupled to the light curing device; and

FIG. 5 is a view illustrating the process of detecting a crack on a tooth using the light curing device to which the tooth crack observation tip of the present invention is coupled.

### DESCRIPTION OF REFERENCE NUMERALS

| 10: light curing device | 11: light curing body |
|---|---|
| 13: coupling pipe | 13a: coupling hole |
| 15: input button | 17: blue LED |
| 20: light guide tip | 21: light guide pipe |
| 23: light diffusion pipe | |
| 100: tooth crack observation tip | |
| 110: tip body | 111: insertion pipe |

113: guide pipe coupling portion
115: finger insertion recess
117: insertion hole
120: crack observation guide pipe
130: light conversion filter

### BEST MODE

Exemplary embodiments of the present invention will be described with reference to the accompanying drawings in order to promote a better understanding of the present invention. The embodiments of the present invention may be modified in many different forms, and the scope of the invention should not be limited to the embodiments set forth herein. Rather, these embodiments are provided so that this disclosure will be thorough and complete, and will fully convey the concept of the invention to those skilled in the art. Therefore, in the drawings, the shapes or the like of the elements may be exaggerated for clarity. It is to be noted that the same reference numerals will be used to designate the same components in each drawing. A detailed description of known functions and configurations incorporated herein will be omitted when the same may obscure the subject matter of the present invention.

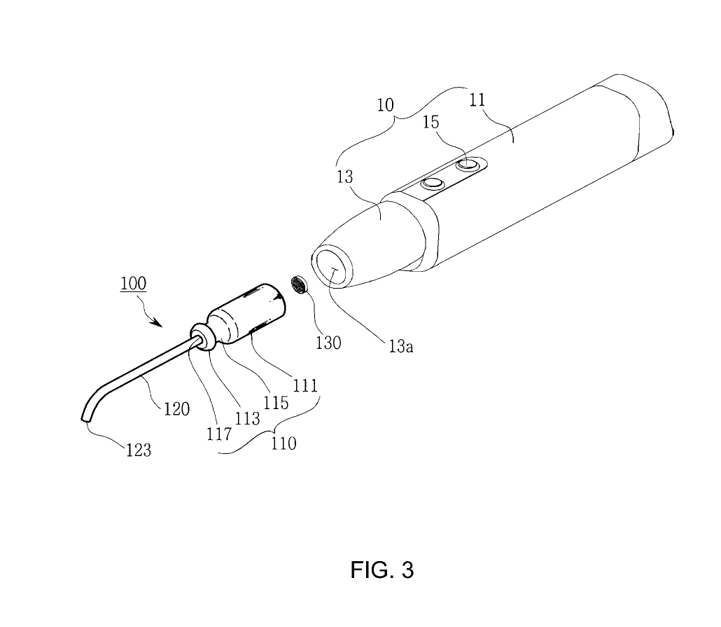

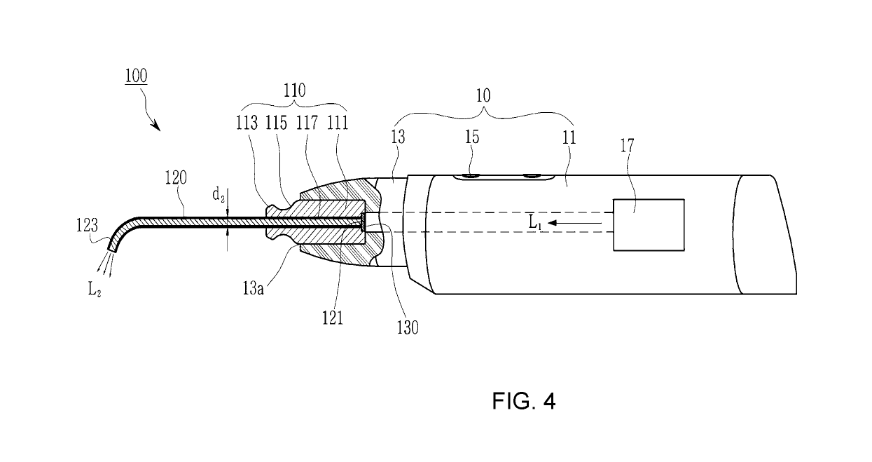

FIG. 2 is a view illustrating the state in which a tooth crack observation tip 100 according to the present invention is coupled to a light curing device 10, and FIG. 3 is a view illustrating the process of coupling the tooth crack observation tip 100 to the light curing device 10.

As shown in FIGS. 2 and 3, the tooth crack observation tip 100 according to the present invention is used in the state of being separably coupled to the light curing device 10. When photopolymerization is needed, the light curing device 10 is used in the state in which a light guide tip 20 is coupled to a coupling pipe 13, and when crack observation is needed, the light curing device 10 is used in the state in which the light guide tip 20 is separated from the coupling pipe 13 and the tooth crack observation tip 100 of the present invention is coupled to the coupling pipe 13.

That is, it is possible to perform both the photopolymerization and the crack observation using a single light curing device 10.

The tooth crack observation tip 100 of the present invention is configured to convert blue light L1 emitted from the light curing device 10 into white light L2 and to guide the white light L2 into an oral cavity, thereby enabling a dental operator to detect the presence of dental caries.

The tooth crack observation tip 100 includes a tip body 110, which is coupled to the light curing device 10, a crack observation guide pipe 120, which extends a predetermined length from the front end of the tip body 110 and guides white light L2, and a light conversion filter 130, which converts blue light L1 generated in the light curing device 10 into white light L2.

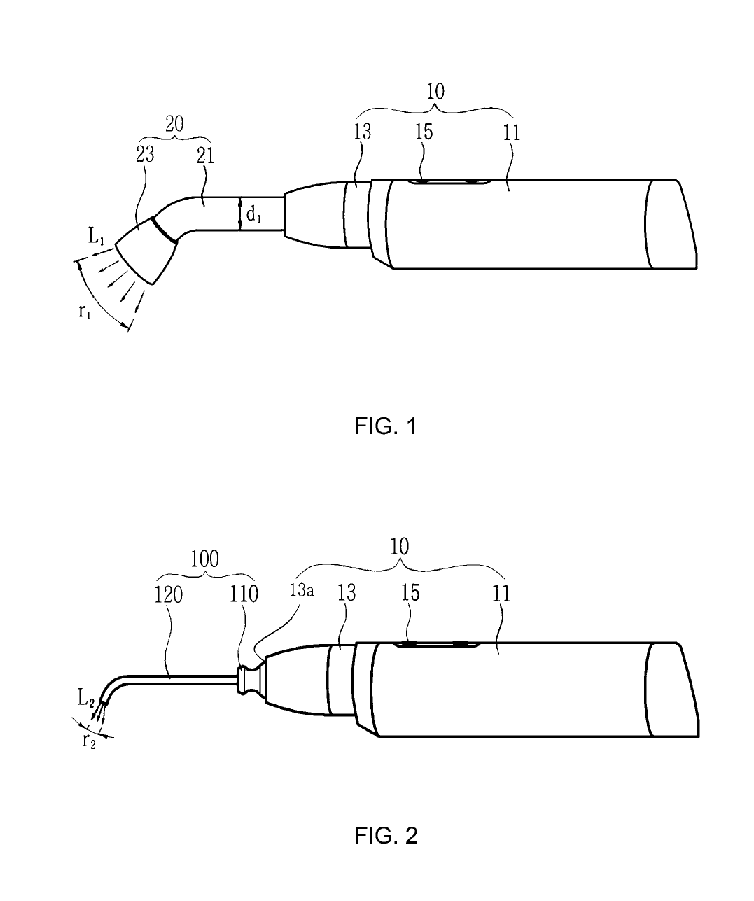

The tip body 110 is inserted into the coupling pipe 13 of the light curing device 10. The coupling pipe 13 has therein a coupling hole 13a, into which a light guide pipe 21 of the light guide tip 20 (see FIG. 1) is inserted. The tip body 110 is formed to have an outer diameter that corresponds to the inner diameter of the coupling hole 13a.

As shown in FIGS. 3 and 4, the tip body 110 is inserted into the coupling hole 13a and is physically fixed to a light curing body 11. The tip body 110 includes an insertion pipe 111, which is inserted into the coupling hole 13a, a guide pipe coupling portion 113 formed at the front end of the insertion pipe 111, a finger insertion recess 115, which is depressed to a predetermined depth from the outer circumferential surface of the insertion pipe 111 so as to allow the fingers of a dental operator to be inserted thereinto, and an insertion hole 117, which is formed to penetrate the insertion pipe 111 so as to allow the crack observation guide pipe 120 to be inserted thereinto.

The insertion pipe 111 is formed to have an outer diameter that corresponds to the inner diameter of the coupling hole 13a. Since the insertion hole 117 is formed to penetrate the insertion pipe 111, the crack observation guide pipe 120 is stably supported by the insertion pipe 111.

The finger insertion recess 115 is depressed to a predetermined depth from the outer circumferential surface of the insertion pipe 111 so that a dental operator can easily couple or separate the tooth crack observation tip 100 to or from the light curing device 10. That is, a dental operator is capable of inserting or separating the tip body 110 into or from the light curing device 10 by inserting the fingers into the finger insertion recess 115.

The crack observation guide pipe 120 is inserted into the tip body 110 and guides light generated from the light curing device 10 to an oral cavity. The crack observation guide pipe 120 is formed of glass fiber in order to guide light.

As shown in FIG. 4, the rear end 121 of the crack observation guide pipe 120 is inserted into the tip body 110. The light conversion filter 130 is coupled to the rear end 121. The front end 123 is inserted into the oral cavity, and white light L2 is emitted toward a tooth through the front end 123.

Unlike the light guide tip 20 that guides light in a wide range, the crack observation guide pipe 120 serves to focus light on a specific potion so that the interior of a tooth is illuminated by white light L2, thereby enabling a dental operator to detect the presence of dental caries with the naked eye. To this end, the crack observation guide pipe 120 has a diameter d2 that is smaller than the diameter d1 of the light guide pipe 21 of the light guide tip 20 depicted in FIG. 1, for example, that ranges from a twentieth to a tenth of the diameter d1 of the light guide pipe 21.

Accordingly, white light L2 is intensively emitted in a relatively narrow range r2 through the front end of the light guide pipe 21.

The light conversion filter 130 converts blue light L1, generated from a blue LED 17 provided inside the light curing device 10, into white light L2. The light conversion filter 130 is coupled to the rear end 121 of the crack observation guide pipe 120, which is inserted into the light curing device 10.

The light conversion filter 130 is embodied by a glass plate having a predetermined thickness, and the two opposite surfaces of the glass plate are colored red and green, respectively. The blue light L1 generated from the blue LED 17 is converted into white light L2 through combination with the red color and the green color.

The blue light L1 is converted into white light L2 while passing through the light conversion filter 130, and the white light L2 is guided by the crack observation guide pipe 120 and is emitted to the oral cavity.

As described above, the light conversion filter 130 may be formed separately, and may be coupled to the rear end 121 of the crack observation guide pipe 120, or may be formed integrally with the crack observation guide pipe 120 as needed. That is, the two opposite surfaces of the rear end 121 of the crack observation guide pipe 120 may be colored red and green, respectively, so as to convert blue light L1 into white light L2.

The process of using the tooth crack observation tip 100 according to the present invention configured as described above will now be described with reference to FIGS. 1 to 5.

A dental operator uses the light curing device 10 in order to harden resin or the like in the oral cavity of a patient. At this time, as shown in FIG. 1, the light curing device 10 is used in the state in which the light guide tip 20 is coupled to the front end of the light curing device 10. The light curing device 10 emits blue light L1 toward a tooth in order to harden resin.

When it is required to detect the presence of dental caries of the patient while using the light curing device 10, the dental operator, as shown in FIG. 3, separates the light guide tip 20 from the coupling pipe 13 of the light curing device 10 and couples the tooth crack observation tip 100 of the present invention to the coupling pipe 13 of the light curing device 10.

The tip body 110 of the tooth crack observation tip 100 is inserted into the coupling hole 13a and is fixed to the light curing device 10. At this time, the dental operator inserts the fingers into the finger insertion recess 115 formed in the tip body 110, and can thus couple the tooth crack observation tip 100 to the light curing device 10 easily.

As shown in FIG. 2, in the state in which the tooth crack observation tip 100 is coupled to the light curing device 10, when power is applied to the light curing device 10 by manipulation of an input button 15, as shown in FIG. 4, the blue LED 17 emits blue light L1.

The blue light L1 is converted into white light L2 while passing through the light conversion filter 130, which is coupled to the rear end of the tip body 110, that is, the rear end 121 of the crack observation guide pipe 120. The white light L2 is guided by the crack observation guide pipe 120 and is emitted to the oral cavity through the front end 123.

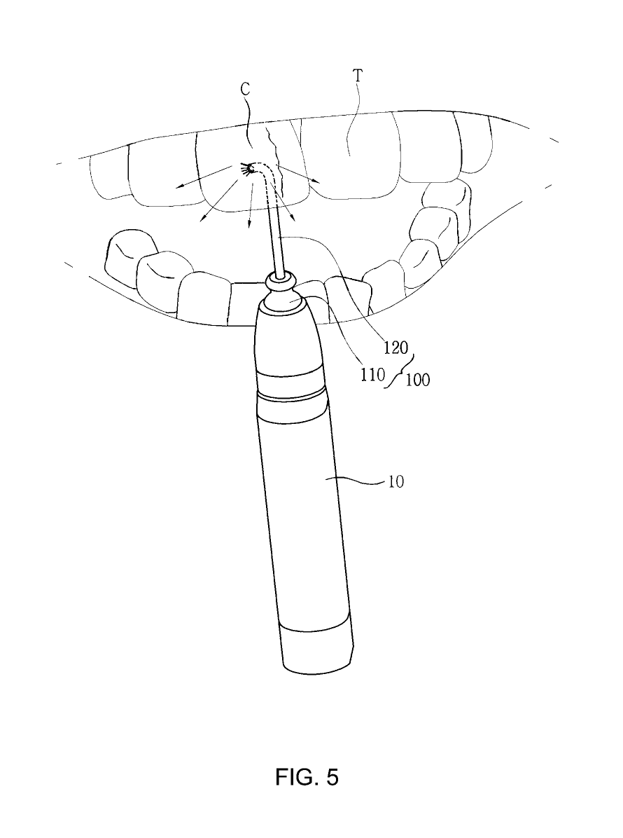

At this time, the white light L2 is emitted in a relatively narrow region r2 through the front end 123. As shown in FIG. 5, when the dental operator shines the white light L2 on the backside of the tooth, the corresponding tooth appears transparent, and thus the dental operator is capable of detecting the presence of dental caries or cracks C.

As is apparent from the above description, the tooth crack observation tip according to the present invention can be used in the state of being separably coupled to a light curing device. As a result, it is possible to realize both a photopolymerization function and a crack observation function using a single light curing device.

In addition, the tooth crack observation tip of the present invention has a simple structure so as to be easily inserted into or separated from a light curing device by a dental operator, and emits white light in a relatively narrow range, whereby a dental operator is capable of detecting the presence of dental caries with the naked eye.

The exemplary embodiments of the tooth crack observation tip couplable to a light curing device of the present invention described above are merely illustrative, and it will be appreciated by those skilled in the art that various modifications and equivalent other embodiments are possible from the present invention. Therefore, it may be appreciated that the present invention is not limited to the forms mentioned in the above detailed description. Accordingly, the actual technical protection scope of the present invention must be determined by the spirit of the appended claims. Further, it is to be construed that various modifications, equivalents, and substitutions are possible, without departing from the scope and spirit of the invention as disclosed in the accompanying claims.

1. A tooth crack observation tip configured to be separably coupled to a light curing device for generating blue light and guiding the blue light to a light guide tip, the tooth crack observation tip comprising:
a tip body configured to be inserted into a coupling pipe of the light curing device from which the light guide tip has been separated;
a crack observation guide pipe extending a predetermined length from the tip body, the crack observation guide pipe being configured to be inserted into an oral cavity; and
a light conversion filter coupled to a rear end of the tip body, the light conversion filter being configured to convert the blue light into white light,
wherein the light conversion filter is formed by glass having two opposite surfaces that are colored red and green, respectively.

2. The tooth crack observation tip according to claim 1, wherein the tip body is formed to have an outer diameter that corresponds to an inner diameter of the coupling pipe, and has a finger insertion recess depressed to a predetermined depth from an outer circumferential surface thereof so as to allow fingers to be inserted thereinto.

3. The tooth crack observation tip according to claim 2, wherein the crack observation guide pipe is formed of glass fiber, and has a diameter that ranges from a twentieth to a tenth of a diameter of a light guide pipe of the light guide tip.

4. A tooth crack observation tip configured to be separably coupled to a light curing device for generating blue light and guiding the blue light to a light guide tip, the tooth crack observation tip comprising:
a tip body configured to be inserted into a coupling pipe of the light curing device from which the light guide tip has been separated; and
a crack observation guide pipe extending a predetermined length from the tip body, the crack observation guide pipe being configured to be inserted into an oral cavity,
wherein the crack observation guide pipe has a rear end that is coupled to the tip body, the rear end having two opposite surfaces that are colored red and green, respectively, so as to convert the blue light into white light.
